(19) United States
(12) Patent Application Publication
(10) Pub. No.: US 2005/0021237 A1
(43) Pub. Date: Jan. 27, 2005

(54) Method and apparatus for evaluating new chemical entities

(76) Inventors: Asher Daniel Schachter, Needham, MA (US); Marco Francesco Ramoni, Boston, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/881,322

(22) Filed: Jun. 30, 2004

Related U.S. Application Data
(60) Provisional application No. 60/484,752, filed on Jul. 3, 2003.

(51) Int. Cl. G01N 33/50

(52) U.S. Cl. 702/19

(57) ABSTRACT

A method for predicting the success of a new chemical entity, including the steps of providing a signal related to the new chemical entity, providing a therapeutic index, providing a conditional probability table, providing a prior probability distribution, providing a prior N, and calculating a posterior probability distribution for the new chemical entity. An apparatus for predicting the success of a new chemical entity including an input for a signal related to the new chemical entity, a therapeutic index, a conditional probability table, a prior probability distribution, a prior N, and a processor calculating a posterior probability distribution for the new chemical entity.

### CROSS-REFERENCE TO RELATED APPLICATIONS

This application claims priority to and the benefit of U.S. Provisional Patent Application Ser. No. 60/484,752, filed Jul. 3, 2003, the disclosures of which are incorporated herein by reference.

### GOVERNMENT SUPPORT

Work described herein was supported by Federal Grant No. NIH K23 RR-16080, awarded by the National Institute of Health to Children's Hospital. The Government has certain rights in the invention.

### TECHNICAL FIELD OF THE INVENTION

This invention relates generally to the field of chemical analysis and more specifically to the development of new chemical entities using a Bayesian Belief Network.

### BACKGROUND

The USA is known as a world-leader in innovation. The drug development domain is an excellent example of America's innovative potential, with many breakthrough medications having been discovered and developed in the USA. This degree of innovation requires consistently huge research and development expenses, and much of this cost is borne by patients and their insurance plans.

A recent analysis by the Tufts Center for the Study of Drug Development estimates that the cost of developing a single new chemical entity (hereinafter “NCE”) into a successful therapeutic agent is $802 million (in 2000 dollars). The clinical phase costs estimated at $467 million and the “time costs” related to the length of time from Investigational New Drug (hereinafter “IND”) approval to New Drug Application (hereinafter “NDA”) marketing approval making up the difference. Although, the $802 million figure is dependent on the proportion of NCEs that fail during the clinical trial development phase.

Compounding this problem is the relatively recent adoption of combinatorial chemistry and high-throughput screening for potential NCEs, significantly increasing the number of early-phase NCEs under consideration for further costly development in human clinical trials. Despite the recent explosion of potential new drugs, the annual rate of NDA approval hit a 5-year low in 2002, with only 18 NDA approvals, compared to 30, 35, 27, and 24 in 1998, 1999, 2000, and 2001 respectively. The only recent improvements in the drug development process are the decreases in mean residence time (the time between IND and NDA approval) by 1.5 years and in median time to research abandonment by 0.8 years, suggesting that drug developers are making faster decisions regarding research failures.

The drug development process consists of several phases and milestones: pre-clinical studies, IND approval, clinical trial phases I/II/III, NDA approval, and phase IV (post-marketing surveillance for idiosyncratic adverse events and potential alternate indications). The patent life of a given NCE typically begins at the time of IND approval and lasts for 20 years, but financial return does not commence until NDA approval is granted and may be short-lived if competitors release similar agents.

Accordingly, it is in the pharmaceutical industry's interest to terminate failures early, and to accomplish successful development phases as quickly as possible without compromising the quality of the clinical trials. This is a delicate balance between financial constraints, proper conduct of clinical trials and good clinical practices, and ensuring that regulatory requirements for approval will be met.

In light of this, the distinction between the innovative development of an NCE and the development of more efficient medications is noteworthy. The latter involves improving on already successful medications by (any or all of) reducing toxicity, increasing potency, reducing the dosing schedule, or by changing to an easier route of administration. Improving a successful agent's efficiency is clearly not as risky as is the development of an NCE, and is rarely a major source of lost revenue.

Analyses of drug development failure consistently reveal that safety, toxicity and economics are the three most important causes of drug failure. Pharmacoeconomic modeling is a vastly different domain compared to the clinical trial domain of the approach described herein, and is beyond the scope of this method. However, the impact of safety and toxicity on NCE failure is significant. The cost of an NCE that will ultimately fail is directly proportional to the length of time between IND approval and termination of development. It follows that earlier termination of NCEs destined for failure results in significantly more savings with the added benefits of limiting patient exposure to potentially unsafe and/or ineffective investigational agents, as well as freeing up clinical trial resources for other more promising agents in the development pipeline.

Analyses of the distribution of research terminations by clinical phase have shown that over 60% of terminations occur during phases II and III; that is, later in the drug development process. Also, because the later phases are more costly, earlier termination of even a fraction of later phase failures results in a factoring of savings: terminating only 5% of all phase III clinical failures in Phase I would reduce out-of-pocket clinical costs by 5.5-7.1%. However, over-zealous termination of NCEs will impede the development of innovative, breakthrough therapies. The decision process must balance the cost of terminating what would be a successful NCE against allowing an eventual failure to proceed through phase III. Pharmacovigilance is a difficult and risky task.

### SUMMARY OF THE INVENTION

In one aspect, the invention is a method for predicting the success of a new chemical entity. The method includes the steps of providing a signal related to the new chemical entity, providing a therapeutic index for the new chemical entity, and providing a conditional probability table for the new chemical entity. Additionally, the method includes the steps of providing a prior probability distribution for the new chemical entity, providing a prior N for the new chemical entity, and calculating a posterior probability distribution for the new chemical entity.

Various other embodiments of this aspect of the invention include the following features. In various embodiments of the method the signal is human clinical trial data, in vivo trial data, and in vitro trial data. The therapeutic index in the method, in one embodiment, is a vital organ therapeutic index and a disease therapeutic index. The conditional probability table that is provided is an efficacy conditional probability table and a safety conditional probability table. The posterior probability distribution in the method is a clinical success posterior probability distribution, an efficacy posterior probability distribution, and a safety posterior probability distribution. When the posterior probability distribution is an efficacy posterior probability distribution that distribution is in response to the pharmacogenomic profile of a patient.

In another aspect the invention is an apparatus for predicting the success of a new chemical entity. The apparatus includes an input for a signal related to the new chemical entity, a therapeutic index for the new chemical entity, and a conditional probability table for the new chemical entity. Additionally, the apparatus includes a prior probability distribution for the new chemical entity, a prior N for the new chemical entity, and a processor calculating a posterior probability distribution for the new chemical entity.

Various other embodiments of this aspect of the invention include the following features. In various embodiments of the apparatus, the signal is human clinical trial data, in vivo trial data, and in vitro trial data. The therapeutic index in the apparatus is a vital organ therapeutic index and a disease therapeutic index. The conditional probability table is an efficacy conditional probability table and a safety conditional probability table. In one embodiment of the apparatus, the posterior probability distribution is a clinical success posterior probability distribution, an efficacy posterior probability distribution, and a safety posterior probability distribution. Additionally, in one embodiment the posterior probability distribution is in response to the pharmacogenomic profile of a patient.

In yet another aspect, the invention is a method for reaching a termination decision regarding a new NCE. In one embodiment, a Bayesian Network is used to reach the termination decision. In another embodiment, the terminal decision is based on a posterior probability of success. In another embodiment, the specific domain of interest for the Bayesian Network is limited to a single NCE. In yet another embodiment, the termination decision is evaluated based upon at least one of the safety, efficacy and clinical success of the NCE. In another aspect this method further includes the steps of providing a signal related to the new chemical entity, providing a therapeutic index for the new chemical entity, and providing a conditional probability table for the new chemical entity. Additionally, the method includes the steps of providing a prior probability distribution for the new chemical entity, providing a prior N for the new chemical entity, calculating a posterior probability distribution for the new chemical entity, and using the posterior probability to reach an NCE termination decision.

### BRIEF DESCRIPTION OF THE FIGURES

These and other aspects of this invention will be readily apparent from the detailed description below and the appended drawings, which are meant to generally illustrate and not to limit the invention, and in which:

FIG. 1 is an example of a 3-layer Bayesian Belief Network known to the prior art.

FIG. 2A is a Bayesian Belief Network of clinical variables believed to be relevant to clinical success for a New Chemical Entity according to an illustrative embodiment of the invention.

FIG. 2B is a Bayesian Belief Network of clinical variables believed to be relevant to clinical success for an New Chemical Entity, with pharmacogenomics added as a leaf variable under Efficacy and Safety according to an illustrative embodiment of the invention.

FIG. 3 is a schematic diagram of how Therapeutic Class and New Chemical Entity Source relate to Clinical Success according to an illustrative embodiment of the invention.

FIG. 4 is a flowchart wherein the prior probability of Clinical Success is determined according to an illustrative embodiment of the invention.

FIG. 5 is a schematic diagram of an algorithm demonstrating the overlap function and the life saving preference are utilized to determine the Conditional Probability Tables for the Safety and Efficacy nodes according to an illustrative embodiment of the invention.

FIG. 6A is a graph showing the logistic sigmoid functions that are used to approximate the P(TI | Safety=T) CPT values from the TI values according to an illustrative embodiment of the invention.

FIG. 6B is a graph showing the logistic sigmoid functions that are used to approximate the P(TI | Safety=F) CPT values from the TI values according to an illustrative embodiment of the invention.

FIG. 7 is a group of linear functions demonstrating how the modified signal:noise ratio value is utilized to approximate P(signal|Efficacy) for a series of randomly-generated New Chemical Entity and control means and variances according to an illustrative embodiment of the invention.

FIG. 8 is a diagram depicting a method for constructing leaf node Conditional Probability Tables according to an illustrative embodiment of the invention.

FIG. 9 is a diagram depicting an overview method according to an illustrative embodiment of the invention.

FIG. 10 is a graphic user interface representation illustrating the prior and posterior probability distributions for Clinical Success for the fictional antineoplastic agent, CurOnc according to an illustrative embodiment of the invention.

FIG. 11 is a graphic user interface representation illustrating the prior and posterior probability distributions for Safety and Efficacy for the fictional antineoplactic agent, Cur Onc according to an illustrative embodiment of the invention.

FIG. 12 is a graphic user interface representation illustrating the effect of selecting the “Not Life Saving” option on the posterior probability distribution for Clinical Success for the fictional antineoplastic agent, CurOnc according to an illustrative embodiment of the invention.

FIG. 13 is a graphic user interface representation illustrating the effect of setting the prior bias to “optimistic” on the prior and posterior probability distribution for Clinical Success for the fictional antineoplastic agent, CurOnc according to an illustrative embodiment of the invention.

FIG. 14 is a graphic user interface representation illustrating the prior and posterior probability distributions for Clinical Success for LY203638 (rhAPC) according to an illustrative embodiment of the invention.

FIG. 15 is a graphic user interface representation illustrating the prior and posterior probability distributions for Safety and Efficacy for LY203638 (rhAPC) according to an illustrative embodiment of the invention.

FIG. 16 is a graphic user interface representation illustrating the effect of setting the prior bias to “optimistic” on the prior and posterior probability distribution for Clinical Success for LY203638 (rhAPC) according to an illustrative embodiment of the invention.

### DETAILED DESCRIPTION OF THE INVENTION

The presently preferred and alternative embodiments of the invention, including the best mode for practicing the invention known at this time, are now described in detail in connection with the accompanying drawings. It is, however, expressly noted that the present invention is not limited to these embodiments, but rather the intention is that modifications that are apparent to the person skilled in the art and equivalents thereof are also included.

In part, the methods and apparatus disclosed herein relate to a Bayesian Belief Network (hereinafter “BBN”) method (called Pharminator), for calculating the posterior probability that a specific NCE will succeed or fail. The success or failure determination can be based on at least on of: (1) prior data regarding success rates for NCEs of the same therapeutic class and source, and (2) the NCE's therapeutic indices, in vitro | in vivo proof of concept data, and proof of concept data in humans from Phase I and early Phase II studies. The main distinction between Pharminator and previously described methods is that Pharminator focuses on evaluating a specific NCE and determining if it warrants further consideration in light of certain parameters.

Other methods have taken more of a population-based analysis approach, yielding valuable data on overall success rates, but not really addressing the needs of drug developers concerned with the termination decision for a single, specific NCE. Additionally, in various embodiments Pharminator includes a graphic user interface representation that facilitates executing the Bayesian network approaches and NCE data manipulation techniques described herein.

Other Bayesian approaches described in the literature differ from Pharminator with respect to the domain to which Bayes theorem is applied. Published Bayesian approaches to pharmacovigilance compare the benefits of Bayesian statistics over frequentist approaches and focus on the utilization of Bayesian statistics for the analysis of clinical trial data which is in turn used to define “stopping boundaries” (explored in detail below). Bayesian theory has also been used to facilitate drug development-related tasks such as determining clinical trial sample size and designing clinical trials. Yet another proposed use for Bayes theorem is as an alternate approach to utilizing population pharmacokinetic data to predict toxicity in ongoing clinical trials. These are all clearly different tasks from the aim of this method, which is neither concerned with the long-standing Bayesian-frequentist debate, nor with the utilization of Bayes theorem to analyze clinical trial results. Pharminator is specific to individual NCEs rather than individual patients or individual studies, and is much more broad in scope in that it attempts to predict safety, efficacy and NCE clinical success for a specific NCE in question.

Currently, a review of the literature for decision analytic approaches to pharmacovigilance yields several publications of interest. Berry et al adopted a Bayesian decision-theoretic approach to determine “stopping boundaries” for the development of an NCE. Their approach utilizes accumulating information on the NCE's performance to determine at which point the clinical trial's evidence of efficacy is sufficiently negative that the trial should be stopped. The authors argue that if prior data are positive, then one should be willing to tolerate somewhat more negative results in the current clinical trial than if previous evidence is also negative. Given that this manuscript was published in 1988, without the benefit of hindsight of the past 15 years' 70-90% NCE failure rate, the authors' argument is representative of a dangerous and costly assumption.

Namely, that the NCE's clinical trial data can be ignored if it is negative in the context of positive prior data. A counter-argument could demand that the posterior probability distribution should be relied upon to reflect the updated belief that incorporates prior data and the NCE's most current evidence, and that if this posterior probability distribution reveals poor performance, serious consideration should be given to terminating the NCE's development. Another difference between Berry's approach and Pharminator is that Berry's approach focuses on efficacy in isolation. Pharminator utilizes a Bayesian belief network to relate safety and efficacy as independent variables, conditional on the common parent (root) node, clinical success. The root node's prior probability distribution is constructed based upon extensive data on NCE failure rates stratified by therapeutic class and NCE source. Berry et al also state the prior distribution is subjective where the subject is the pharmaceutical company. This forces one to predict the posterior probability for a pharmaceutical company rather than for a specific NCE.

Spiegelhalter et al make a cogent argument for the superiority of Bayesian over frequentist models for the analysis of clinical trial data. The authors promote the use of Bayesian statistics to analyze the outcome of specific ongoing clinical trials. Their argument is not relevant to this method. Pharminator utilizes the NCE's characteristics within the framework of a Bayes network to predict the outcome for a given NCE; clearly a different use of Bayes theorem compared to approach advocated by Spiegelhalter.

Similarly to Spiegelhalter, Johns and Andersen describe the utility of predictive probabilities for interim analyses of phase II and phase III clinical trials. Pharminator focuses on earlier phase decisions so as to avoid costly phase II and phase III trials. Pallay describes the use of Bayes theorem for economically oriented futility analyses of ongoing phase II clinical trials. This is vastly different from the conditional dependencies incorporated into Pharminator, which relates clinical success, efficacy, safety, therapeutic indices and proof of concept data to update prior beliefs pertaining to the NCE's therapeutic class and source. Taken together, the persistently high rate of NCE failures is the strongest evidence that these previously published and widely adopted approaches do not appear to enable drug developers to be sufficiently accurate in their pharmacovigilance decisions.

The present method and apparatus will facilitate improving the efficiency of development of NCEs. The product of this method is an application (Pharminator) to be used by the drug development team at the phase I|early phase IIa time point for a given NCE that has already passed the IND screening process. The user is prompted to answer several key questions about the NCE (detailed below). The answers provide information regarding which prior probabilities and conditional probability tables are to be used in the method, as well as the observed data upon which prediction will be made. The output is a numeric and graphical (binomial distribution plot) report of the prior and posterior probability distributions for clinical success, safety and efficacy.

A Bayesian Network (often referred to as a Bayesian Belief Network (BBN) or a ‘Bayes Net’) is defined as a directed acyclic graph encoding assumptions of conditional independence, with stochastic variables represented as nodes within the network, and inter-variable dependencies represented as inter-nodal links. In addition to a graphical model, a BBN also requires the definition of certain parameters for probabilistic inference purposes. Therefore, it is necessary to specify the conditional probability distribution for each node.

For distributions with a binary outcome (i.e. 2 states), the conditional probability distribution can be represented as a 2×2 conditional probability table (CPT). These tables specify the probabilities that the node is in state (0,1) given that its parent is in state (0,1). The CPT for the top (root) node, which has no parent node, is that root node's prior probability distribution. Assuming conditional independence, and utilizing the chain rule of probability, the joint probability for a network consisting of a root node, R, that has n child nodes, C₁, C₂, . . . Cₙ can be calculated:
P(R, C₁, C₂, . . . Cₙ)=P(R)*P(C₁|R)*P(C₂R) * . . . P(CₙR)

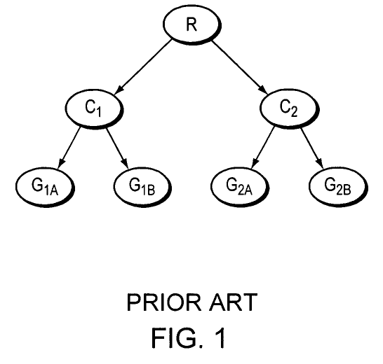

FIG. 1 is a schematic illustration of a 3-layer Baysian Belief Network known to the prior art in which the root node (R) has 2 child nodes (C) and each child node has 2 child nodes (i.e. that are “grandchildren” (G) to the root node), the joint probability for the network can be calculated as in Formula 1 below:

Formula 1: Calculation of the Joint Probability Over a Bayesian Belief Network

$$\begin{matrix}P ( R , {C}_{1} , {C}_{2} , {G}_{1 A} , {G}_{1 B} , {G}_{2 A} , {G}_{2 B} ) = P ( R ) * P ( {C}_{1} \text{\vert} R ) * P ( {C}_{2} \text{\vert} R ) * P ( {G}_{1 A} {\text{\vert C}}_{1} ) * \\ P ( {G}_{1 B} \vert {C}_{1} ) * P ( {G}_{2 A} \text{\vert} {C}_{2} ) * P ( {G}_{2 B} \text{\vert} {C}_{2} ) \\ = {{\prod }_{i = 0}^{n}}_{for n nodes} P ( {node}_{i} \text{\vert} parent )\end{matrix}$$

The inner-layer nodes for this network are referred to as hidden nodes, and the lowest layer nodes as leaf nodes. Calculating the Bayesian posterior probability distribution of the root node given that the leaf nodes are in a specified state is a ratio of the sum of joint distributions. For the example network described above, calculating the probability that the root node is in state ‘F” (false), given that all 4 leaf nodes are in state ‘T” (true) is achieved as follows:

Formula 2: Example Calculation of Root Node Posterior Probability

$$P ( R = F \vert {C}_{1} , {C}_{2} , {G}_{1 A} = T , {G}_{1 B} = T , {G}_{2 A} = T , {G}_{2 B} = T ) \\ = \frac{{\sum }_{v ∈ \{ T , F \}}^{} {\sum }_{w ∈ \{ T , F \}}^{} P ( R = F , {C}_{1} = v , {C}_{2} = w , {G}_{1 A} = T , {G}_{1 B} = T , {G}_{2 A} = T , {G}_{2 B} = T ) }{{\sum }_{x ∈ \{ T , F \}}^{} {\sum }_{v ∈ \{ T , F \}}^{} {\sum }_{w ∈ \{ T , F \}}^{} P ( R = x , {C}_{1} = v , {C}_{2} = w , {G}_{1 A} = T , {G}_{1 B} = T , {G}_{2 A} = T , {G}_{2 B} = T )}$$

Each of the joint probability distributions in Formula 2 can be calculated utilizing Formula 1.

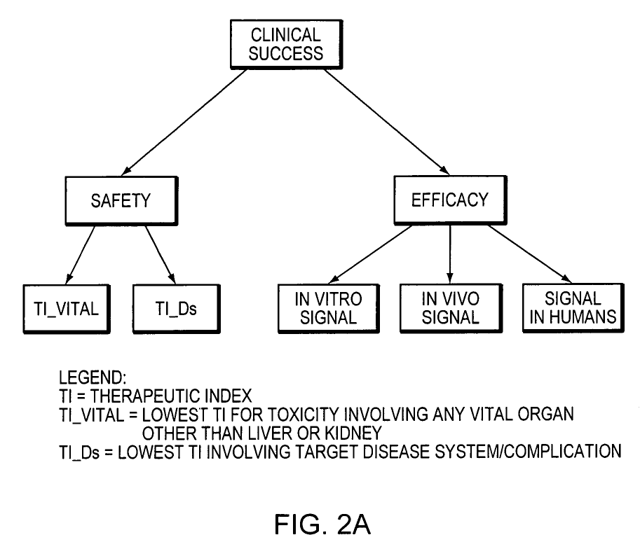

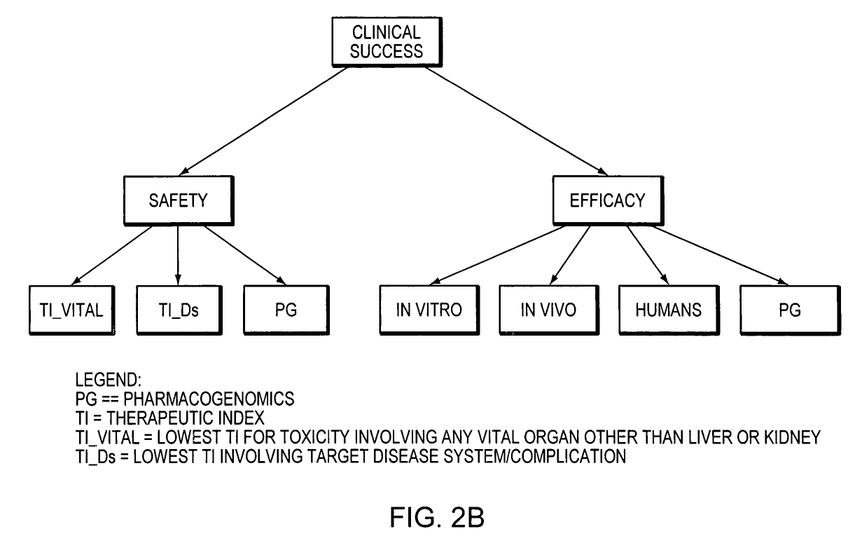

In this way, Bayes theorem can be applied to a given BBN. At the core of the Pharminator algorithm is a BBN encompassing relevant clinical variables in pharmacovigilance. FIG. 2A is an illustrative embodiment of a BBN of clinical variables believed to be relevant to clinical success for an NCE, with implicit assumptions of conditional independence. The structure of this network is designed to include only those variables believed to be most critical to predicting NCE failure, based on the literature, the author's training, and consultation with drug development experts.

The downward direction of the arrows encodes the NCE's deep or hidden knowledge within “Clinical Success” that is manifested as “Safety” and “Efficacy”. The knowledge embedded within each of these two hidden nodes is in turn manifested as therapeutic indices or proof-of-concept signals, respectively. This may seem counter-intuitive, since it may seem logical to believe that safety and efficacy cause clinical success (or failure) rather than represent a manifestation of an unknown degree of clinical success. However, the goal of Pharminator is to predict the clinical success inherent in the NCE, rather than to identify causes of clinical success.

The NCE has an inherent true degree of “Safety” and “Efficacy”. A major goal of clinical trials is to determine what these true values are by studying samples and by assuming that the safety or efficacy in the studied samples are accurate estimates of the NCE's true safety and efficacy. The same explanation can be used to justify the links going from Safety to the therapeutic index nodes, and from Efficacy to the signal nodes. It is this representation that allows Pharminator to predict what the NCE's inherent clinical success is based upon the observed therapeutic indices and proof-of-concept signal data. As this is a BBN, the assumption of conditional independence is required. Therefore, TI_Vital is assumed to be independent of TI_Disease (see FIG. 2A and Section B2 for definitions) conditional on the common parent node, Safety. The same is true for the in vitro, in vivo and human signal nodes, and their common parent, Efficacy.

FIG. 2A is designed to be a “best guess” representation of those variables deemed most important in predicting clinical success. Other embodiments of Pharminator will encode a BBN with slightly different leaf nodes, based upon accumulated data on NCE failure specific to therapeutic classes. Also absent from the BBN is a representation of idiosyncratic severe adverse events which are unpredictable, by definition. Other embodiments of Pharminator include pharmacogenomic markers of adverse events and drug resistance, as depicted in FIG. 2B. FIG. 2B is an illustrative embodiment of a BBN of clinical variables believed to be relevant to clinical success for an NCE, with pharmacogenomics added as a leaf variable under Efficacy and Safety.

As used herein, the term “clinical success” refers to an NCE that is still on the market 1 year after NDA approval.

As used herein, the term “efficacy” refers to an NCE that produces a “sufficient” degree of change in a surrogate or true marker, compared to control (placebo or current gold standard therapy). As used herein, the term “sufficient” degree of change depends on (1) the clinical indication and (2) the development phase (during Phase II, the signal need not be statistically significant, while Phase III studies must show statistical significance in at least two separate trials). Certain indications may require only modest effect from an NCE in order to be successful (e.g. acute, relatively benign disorders), while others may require extreme effects (e.g. life-saving therapies).

As used herein, the term “life-saving” refers to an NCE the disease for which it is indicated is fatal and, there are no alternate life-extending therapies. The Pharminator utilizes the life-saving status of the NCE to determine the influence that the Safety data will have on the calculation of the posterior probability of Clinical Success. The assumption is that a higher degree of toxicity is tolerated for an NCE that is truly life-saving, as defined above, thereby making the probability of clinical success largely dependent on efficacy. For example, an NCE that truly extends life expectancy but causes acute renal failure may still have a high probability of success because it is assumed that the initiation of dialysis is preferable to death. This assumption is open to argument from the point of view of quality of life issues, since truly curative therapies for lethal disorders are rare, however assuming that supportive therapies are preferable to death is reasonable.

As used herein, the term “marker” refers to an indicator of response to therapy. A surrogate marker is a marker that is not directly or primarily involved in the pathogenesis of the disease, whereas a true marker is primarily integral to the disease mechanism. An example of a surrogate marker is the CD4 count in HIV. A true marker for HIV is viral load.

As used herein, the term “acquired NCE” refers to an NCE that a pharmaceutical company has licensed-in from another company, such as a biotechnology firm, or that has been acquired from some other source. As used herein, the term “self-originated NCE” refers tp an NCE for which the initial pre-clinical development occurred within the same pharmaceutical company that will assume responsibility for conducting clinical trials. The prior probability of success differs significantly between acquired and self-originated NCEs. Not surprisingly, NCEs that have undergone initial clinical testing abroad (and demonstrate potential effectiveness in humans) are more likely to succeed.

Pharminator gives the user the option of selecting whether the prior probability should be optimistic or pessimistic. As described in detail in “Prior Probabilities and Conditional Probability Tables”, the NCE's intended therapeutic class affects the selection of the NCE's prior probability of clinical success. Prior data on NCE success rates are stratified by therapeutic source, and include the total number of NCEs within each therapeutic class, the fraction of the total NCEs that have failed, and the fraction of the total NCEs that are still under development, for a total of 671 NCEs spanning IND filing dates from 1981 to 1992. However, it should be noted that if an NCE is sufficiently safe and effective, the NCE's posterior probability of success will be high, regardless of the prior probability.

The Tufts Center for the Study of Drug Development's (TCSDD) published reports provide the prior probability of failure based on the current failure rate, as well as the probability of failure assuming that all NCEs still under development are successful (i.e. a more optimistic prior probability of failure). When the user sets Pharminator's prior bias to “pessimistic” (the default setting), the former prior probability is used— i.e. the prior probability of failure based on the current failure rate. When the user sets Pharminator's prior bias to “optimistic”, the latter prior probability is used— i.e. the prior probability of failure based on the assumption that all NCEs still under development will not fail.

As used herein, the term “safety,” when referring to an NCE, is essentially synonymous with toxicity. Every NCE that is not an inert placebo has some degree of “toxicity” in that even the desired effects of an NCE become toxic if a large enough dose is given. An NCE's “safety” is therefore defined as a degree of toxicity that is an acceptable balance against the benefit to the patient. Ultimately, an NCE can only be deemed safe once it has undergone Phase IV post-marketing surveillance. Prior to Phase IV, insufficient numbers of patients have received the NCE such that rare but severe idiosyncratic reactions would not likely be detected.

As used herein, the term “therapeutic class” refers to the organ system affected by the disease process for which the NCE is indicated. This definition of therapeutic class is utilized rather than the more traditional chemical class because prior probabilities of success are known for a total of 671 NCEs, stratified by therapeutic class, and stratifying by chemical class would fractionate the data beyond use with too many categories and too few NCEs in each category. The therapeutic classes included in Pharminator are: Analgesic/Anesthetic, Antimicrobial, Antineoplastic, Cardiovascular, Central Nervous System (CNS), Endocrine, Gastrointestinal (GI), Immunologic, Respiratory, and Miscellaneous. Clearly these are less specific categories than those used by clinicians (e.g. Calcium channel blockers, ACE inhibitors, mono-amine oxidase inhibitors etc.) however, as stated above, there do not appear to be sufficient data to allow for a more specific stratification without fractionating the data beyond utility.

As used herein, the term “therapeutic index” (TI) refers to the ratio of the NCE dose that produces an undesired effect to the NCE dose that produces the desired effect in a proportion of the study population. The numerator is the TDₓ (toxic dose in x % of the population) and the denominator is the EC_{y} (effective dose in y % of the population). Each NCE has several therapeutic indices, depending on the number of specific adverse events (e.g. the TI for hepatotoxicity is different from the TI for nephrotoxicity), the number of specific desired effects (e.g. ACE inhibitors reduce blood pressure and reduce proteinuria), and depending on the definition of the proportion of the study population (i.e. the values of x and y). A larger TI represents a generally safer NCE. A smaller TI will be either too unsafe to be used clinically, or will require very close therapeutic drug monitoring in order to ensure safety (e.g. digoxin). The Pharminator is designed to be inherently pessimistic, given the high rate of NCE failures to date, and the extreme costs associated with these failures. Therefore, the current implementation of Pharminator requires input for two specific TIs: the lowest TI for an undesired effect on any vital organ (brain, heart, lungs, liver, kidney, exocrine pancreas, bone marrow), and the lowest TI for an undesired effect on any organ or system that is already adversely affected by the disease/system for which the NCE is indicated. An example of the latter is retinal toxicity caused by an NCE indicated for the treatment of diabetes mellitus. In other embodiments of the Pharminator, additional or alternate TI variables are added to the method.

One aspect of the invention recognizes the reasons why therapeutic class and NCE source are included in the method as “prior probability modifiers” and not as stochastic variables (nodes) in FIG. 2A. As discussed, “Network Structure and Rationale”, Safety and Efficacy do not “cause” clinical success in the Pharminator method. The Safety and Efficacy nodes (and indeed all child nodes in the method) are in fact manifestations of the inherent degree of clinical success of the NCE. Contrary to this, therapeutic class and NCE source have a direct impact on NCE clinical success. Additionally, while the NCE's true safety, therapeutic indices, efficacy and proof-of-concept signals are not known, the NCE's intended therapeutic class and source are known with certainty. It is therefore nonsensical to represent therapeutic class and NCE source as stochastic variables. The approaches disclosed herein make use of this distinction.

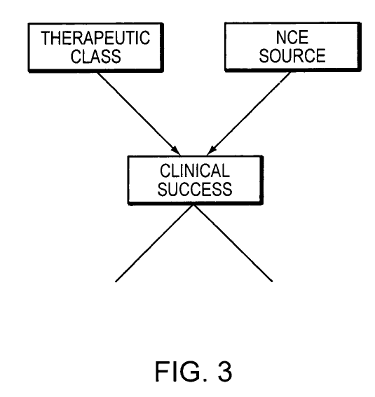

However, FIG. 3 is provided below as an adjunct to FIG. 2A, in order to demonstrate the dependencies between therapeutic class, NCE source and clinical success, and to demonstrate that the noisy-or assumption can be utilized to calculate the prior probability of clinical success from the prior data on NCE failure rates stratified by therapeutic class and NCE source.

FIG. 3 is a schematic illustration of how Therapeutic Class and NCE Source relate to Clinical Success (see also, FIG. 2A). Referring to FIG. 3, the direction of the arrows indicates that Therapeutic Class and NCE Source have a causal effect on Clinical Success. Determining P(Clinical Success | Therapeutic Class, NCE Source) is therefore not a Bayesian posterior probability and the noisy-or assumption is applicable.

### PRIOR PROBABILITY: SOURCE & SELECTION

TCSDD publications are the most extensive, accessible, and reliable source of the prior probability of NCE success (and failure). DiMasi recently analyzed the causes of failure and reported the success rates for 671 NCEs for which INDs were filed between 1981 and 1992. In his report, he provides “current and maximum possible success rates” stratified by therapeutic class for 503 self-originated NCEs. The “current success rate” is the fraction of the number of NCEs (in that class) that have been successful over all NCEs in that class. This is in fact a pessimistic prior because the implicit assumption is that all open NCEs (i.e. NCEs still in development) will fail. The “maximum possible success rate” is the success rate assuming that “all open NCEs will eventually be approved”—an optimistic assumption. DiMasi also provides probabilities of NCE success stratified by NCE source. Pharminator asks the user to indicate the NCE's therapeutic class, NCE source, as well as the user's desired “prior bias”, which may be either pessimistic or optimistic. The prior bias determines which therapeutic class prior probability is utilized: if the user selects “pessimistic” (the default setting), the current success rate is used to calculate the NCE's prior probability of success. Conversely, if the user selects “optimistic”, the maximum possible success rate is used.

Although DiMasi's analysis is quite informative, he did not sub-stratify by therapeutic class and NCE source combinations. In order to allow Pharminator to choose a prior probability that most accurately reflects the NCE's therapeutic class and its source, the algorithm utilizes the noisy-or assumption. Paraphrasing Szolovits, the noisy-or assumption states that the probability that some set of variables causes an outcome equals the probability that at least one of the variables does so. The probability of interest is P(Clinical Success | Therapeutic Class, NCE Source). For an explanation as to why this is not a posterior probability distribution, see section B2 and FIG. 3. Given the noisy-or assumption, the probability of interest can be calculated:
1−P(Clinical Success|Therapeutic Class, Source)=(1−P(Clinical Success|Therapeutic Class))*(1−P(Clinical Success|Source))
Therefore, P(Clinical Success|Therapeutic Class, Source)=1−[(1−P(Clinical Success|Therapeutic Class))*(1−P(Clinical Success|Source))]≈the prior probability,P(Clinical Success) for the NCE in question and P(Clinical Failure)=1−P(Clinical Success) Formula 3: Noisy-Or

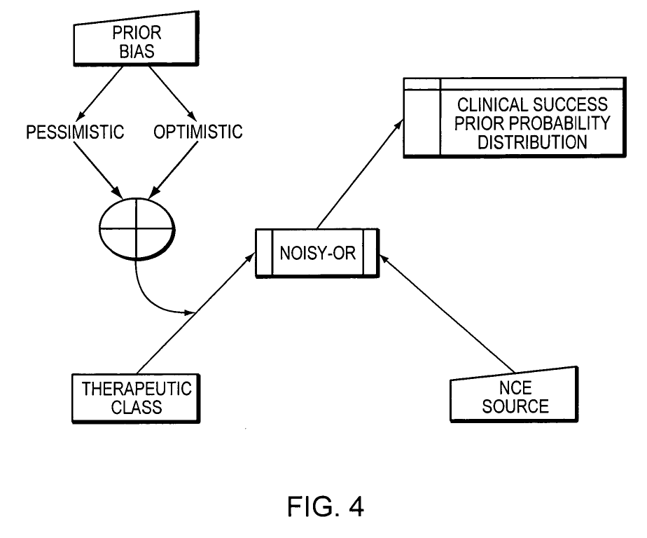

Pharminator utilizes the prior bias selected by the user to determine which prior probability of success to utilize for the selected therapeutic class, then uses this probability along with the probability of success for the selected NCE source to calculate P(Clinical Success|Therapeutic Class, Source), given the noisy-or assumption as in Formula 3 above. This calculated probability is utilized as the prior probability of Clinical Success for the NCE in question. FIG. 4 is an illustrative flowchart of one embodiment of the invention wherein the prior probability of Clinical Success is determined.

### Conditional Probability Tables

As described in section B1, a BBN requires CPTs for each node in order to be utilized for probabilistic inference. Although the data provided in the TCSDD reports is valuable, the format of those reports is not directly applicable to the construction of BBN conditional probability tables. The main limitation of Pharminator is the absence of appropriate conditional probability data and the need to make certain assumptions to allow utilization of the data from the TCSDD published reports. Other embodiments of Pharminator focus on the task of obtaining appropriate data to populate the CPTs. These assumptions will be tested by sensitivity analyses once characteristics and outcomes for specific successful and failed NCEs become available. In the absence of such data, the methods by which the CPTs are currently constructed are described in this section.

DiMasi analyzed the causes of failure for 348 NCEs that were withdrawn from development. It should be noted that NCEs that proceeded through all clinical trial phases but failed to achieve NDA approval are not included in DiMasi's analysis. As well, DiMasi stratified the causes of failure by “primary” cause, thereby not disclosing any degree of overlap—i.e. NCEs that failed primarily due to one reason, but may have also failed for another reason (e.g. an NCE that failed because it was not safe, but was also not very effective). His analysis demonstrated that of a total of 348 NCEs that were terminated, the primary reason for termination was efficacy in 121, safety in 72, economics in 109, and “other” in 46. Since Pharminator is concerned only with safety and efficacy, the probability that safety is the primary cause of failure is 72/(72+121)=0.37, and the probability that efficacy is the primary cause of failure is 121/(72+121)=0.63. Assuming that the proportions of causes of failure are consistent across the withdrawn drugs, the CPT probabilities, P(Safety=F|Clinical Success=F) and P(Efficacy=F|Clinical Success=F), can be calculated by an “overlap” function, as follows:
P(Safety=F|Clinical Success=F)=total number of primary safety failures+(total number of primary safety failures * proportion of primary efficacy failures)=72+(72*121/193)=0.606 Formula 4: “Overlap” Function
P(Efficacy=F|Clinical Success=F)=total number of primary efficacy failures+(total number of primary efficacy failures * proportion of primary safety failures)=121+(121 * 72/193)=0.860

These values (and their respective complement values) occupy the first rows of their respective CPTs.

While the overlap function permits estimation of the first row of each of the Safety and Efficacy CPTs (i.e. P(node=F|parent=F) and P(node=T|parent=F)), currently, there are no adequate, available data for the second rows of the Safety and Efficacy CPTs (P(node=F|parent=T) and P(node=T|parent=T)). For now, these values are currently set as pessimistic estimates. For the Efficacy CPT, the probability P(Efficacy=F|Clinical Success=T), i.e. the probability that an NCE is not efficacious given that it is clinically successful, is logically estimated to be very low. Until data for sensitivity analyses become available, this value is set at 0.01, and its complement, P(Efficacy=T|Clinical Success=T) is therefore 1-0.01=0.99. For the Safety CPT, the probability P(Safety=F|Clinical Success=T), i.e. the probability that an NCE is not safe given that it is clinically successful is also estimated to be low, but likely not as low as for P(Efficacy=F|Clinical Success=T). Until data for sensitivity analyses become available, this value is set at 0.05 for NCEs that are not life-saving, and for NCEs that are life-saving, this value is set at 0.5. This difference is to reduce the influence that TIs have on the posterior probability of clinical success for life-saving NCEs.

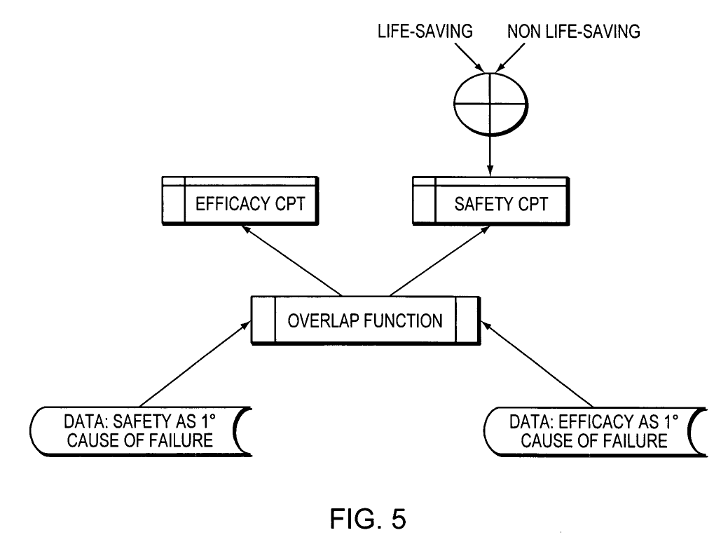

FIG. 5 is a schematic illustration of the construction of the hidden nodes' CPTs. FIG. 5 is a schematic illustration of an algorithm demonstrating one embodiment of the invention wherein the overlap function (Formula 4) and the life-saving preference are utilized to determine the CPTs for the Safety and Efficacy nodes.

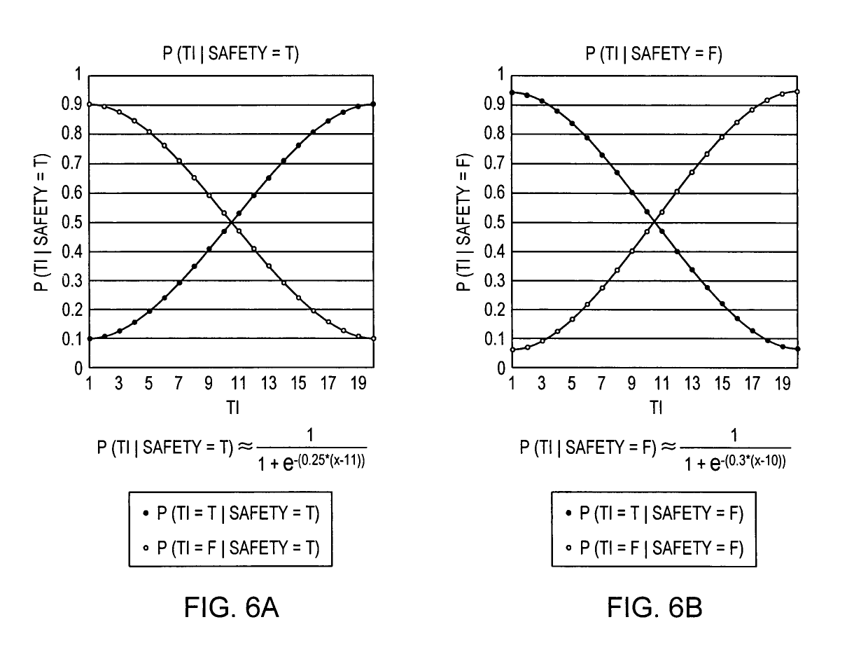

Just as for the hidden nodes (Safety and Efficacy), there are no easily accessible data on therapeutic indices and proof of concept signal data for NCEs that have failed. However, devising models that approximate these relationships is a somewhat less arduous task than for Safety and Efficacy. With respect to the relationship between TI and Safety, it is known that TI is directly proportional to the degree of safety because a larger TI simply means more prescribing “room” between the effective dose and the toxic dose. Most prescription medications have TIs that are in the 8-10 range. Given that TI is a ratio, and that the lowest rational value for a TI is 1, the relationship between TI and safety can be approximated by a logistic sigmoid model (Formula 5, FIG. 6):

### Formula 5: Logistic Sigmoid Function

$$\begin{matrix}P ( T1 \vert Safety ) \approx \frac{1}{1 + {ⅇ}^{- ( s * ( x - i ) )}} \\ x = TI \\ s = s lope \\ i = intercept\end{matrix}$$

The slope and intercept of this model were selected to reflect what is believed to be an accurate approximation of the relationship between TI and P(TI|Safety). FIG. 6A is a graph showing the logistic sigmoid functions that are used to approximate the P(TI|Safety=T) CPT values from the TI values. FIG. 6B is a graph showing the logistic sigmoid functions that are used to approximate the P(TI|Safety=F) CPT values from the TI values.

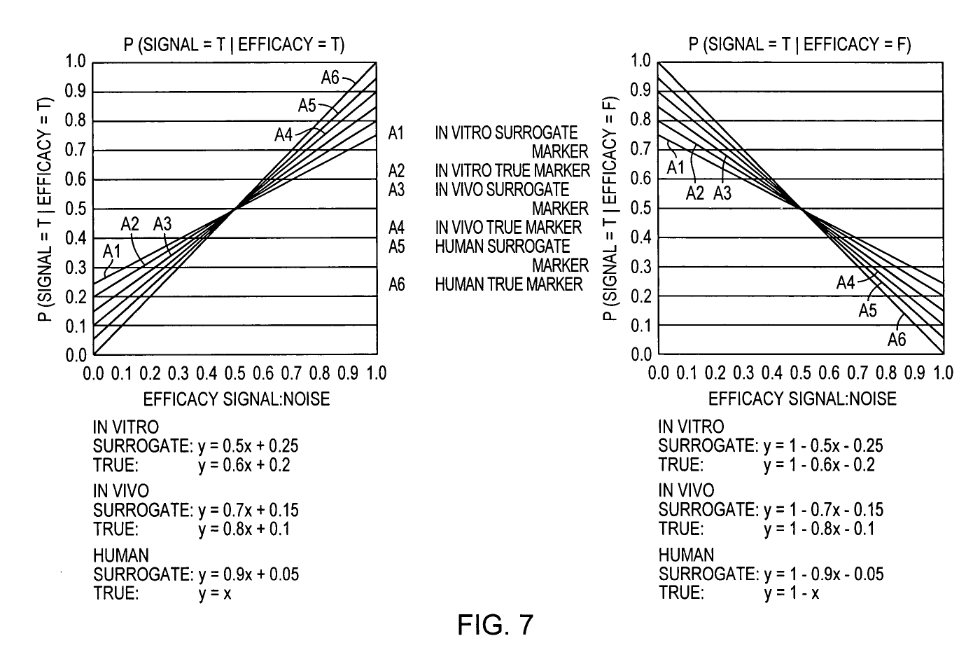

A similar assumption is made for the proof-of-concept signal data in that the quantity of the signal is proportional to the degree of efficacy. In contrast to the sigmoid relationship between TI and safety, the relationship between proof-of-concept signal and efficacy is assumed to be a simple linear function (y=mx+b). However, the signal data must first be transformed to a standardized measure so that different ranges and scales will not influence the interpretation of the signal. For this purpose a modified signal-to-noise ratio is used (Formula 6), requiring the user to enter the mean and variance (S.D.²) for the control and experimental groups, for each experimental environment (in vitro, in vivo—highest-order species, human), as well as whether each signal is a true or surrogate marker. This formula provides a variance-corrected measure of the degree of signal as a value between 0 and 1. The resultant signal-to-noise ratio value is utilized by the linear models to estimate P(Signal|Efficacy) for in vitro, in vivo and human signals, stratified by true and surrogate markers. The slopes (and intercepts) of the linear models are adjusted to reflect differences between in vitro, in vivo and human signals, and between true and surrogate markers (FIG. 7). Specifically, the slope of the function is proportionate to the environment order (in vitro<in vivo<human), and the marker (surrogate<true).

### Formula 6: Modified Signal:Noise Ratio

$$\begin{matrix}Modified signal : noise = \frac{\vert ( \overline{NCE} - \overline{control} ) \vert}{max ( NCE 95 \% UCL , control 95 \% UCL )} \\ UCL = upper confidence limit\end{matrix}$$

FIG. 7 is a group of linear functions demonstrating how the modified signal:noise ratio value is utilized to approximate P(signal|Efficacy) for a series of randomly-generated NCE and control means & variances. The linear functions are stratified by experimental environment (in vitro, in vivo, human), and type of marker (surrogate vs. true marker). The slopes and intercepts are adjusted to reflect what is believed to be an adequate approximation.

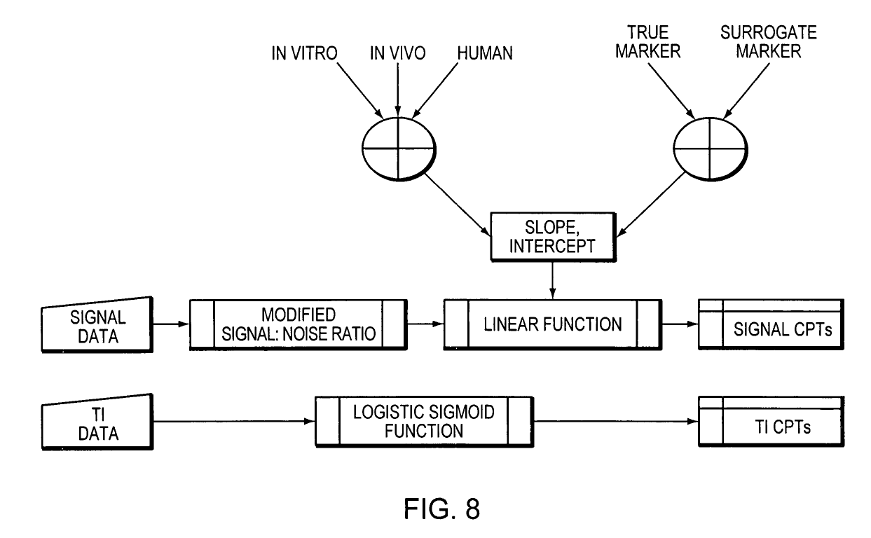

FIG. 8 is an illustrative embodiment of an algorithm for constructing the leaf node CPTs.

The default state for all leaf nodes is “True”, and it is the leaf node CPTs that change in response to the TI and signal data entered by the user. This represents one major departure from BBN methodology: Pharminator's use of the input data to determine the specific CPT to be used for a given set of fixed leaf node states. The acquisition of data on NCEs that have failed will facilitate the modification of the linear and sigmoid functions rather than direct changes to the leaf node CPTs. Justification for this approach is that this can actually work to the advantage of each drug development institution that utilizes Pharminator. Many pharmaceutical companies develop medications in a small number of therapeutic classes and therefore have NCE failure data that is highly specific to that pharmaceutical company's future development projects. Therefore the use of these data to modify Pharminator's CPT functions will result in a company-specific implementation of Pharminator, the predictive accuracy of which will be directly proportionate to the specific company's development history and prior investments in NCE failures (i.e. accuracy proportionate to their losses).

Smaller companies with little or no development history will not have the ability to implement a company-specific implementation, but will benefit from the prior knowledge of the entire industry, excluding confidential and privileged information from other companies. Data from a larger pharmaceutical company will remain exclusive to that specific pharmaceutical company unless that company agrees to allow Pharminator to utilize their data for the benefit of the entire industry, always maintaining confidentiality regarding specific NCEs that have failed.

### Data Input and Output

Pharminator requires the following input from the user: NCE “demographics” including NCE name, therapeutic class, source, and life-saving status; the user's prior bias preference (pessimistic vs. optimistic; default is pessimistic); signal data for each of in vitro, in vivo (highest-order species) and human; the Minimum TI_Vital; and the Minimum TI_Disease.

The signal data are entered for the maximal dose given, regardless of toxicity. Therefore, toxicity (safety) is not taken into account for signal data because Safety and Efficacy are assumed to be independent variables, conditional on the common parent, Clinical Success. The signal nodes' inputs include NCE mean and variance, control mean and variance, and the type of marker: true or surrogate.

The current implementation of Pharminator requires all of these values to be entered in order for the posterior probability to be calculated. Other embodiments of Pharminator perform probability inference on partial nets (i.e. nets that are missing one or more leaf node variables).

With this information, Pharminator selects the appropriate prior probability of clinical success, and calculates the posterior probability distribution for Clinical Success, Safety, and Efficacy. The hidden node “prior” probabilities are required in order to calculate hidden node posterior probabilities. These “prior” probabilities are calculated from the hidden and root nodes° CPTs:
P(Hidden Node)=Σ[P(Hidden Node|Parent Node)*P(Parent Node)] Formula 7: Calculating a Hidden Node's “Prior” Probability

The prior and posterior probability distributions are displayed graphically as binomial distributions. The “n” for the Clinical Success prior probability distribution (“prior N”) is the total number of NCEs from which the prior data were attained. The “n” for the Clinical Success posterior probability distribution (post N) is (prior N+1). This is likewise for the prior N and post N for the Safety and Efficacy probability distributions (see section “Implementation and Examples” for a pictorial demonstration).

### Algorithm

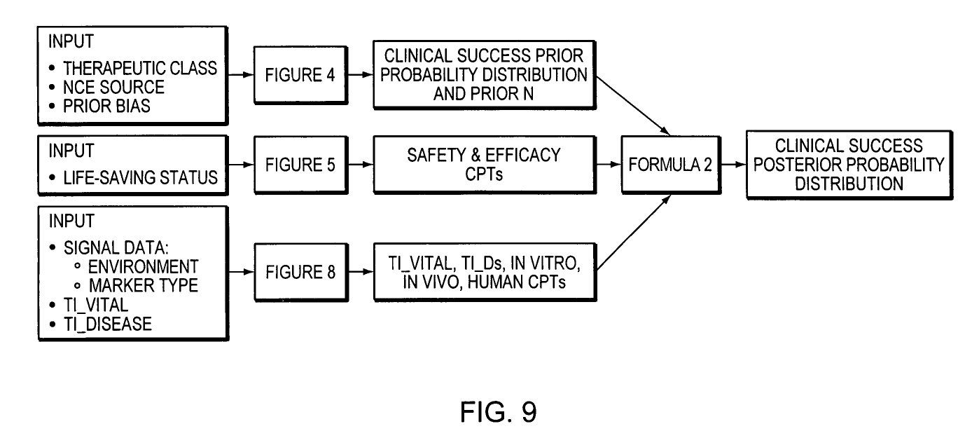

Conglomerating the discussion of the previous sections, including FIGS. 4, 5 and 8 and Formula 2 results in the algorithm shown in FIG. 9. FIG. 9 is a schematic illustration of one embodiment of the invention wherein an overview algorithm is used in conjunction with the invention, combining the components as described.

Implementation

Pharminator is implemented in Java 1.4, using Apple ProjectBuilder v2.1, on Apple OS X.2 Jaguar. An object-oriented, model-view approach was utilized to structure to the program. The accuracy of the BBN was validated against Bayesware Discoverer® (http://bayesware.com).

The methods and systems described herein can be performed in software on general purpose computers, servers, or other processors, with appropriate magnetic, optical or other storage that is part of the computer or server or connected thereto, such as with a bus. The processes can also be carried out in whole or in part in a combination of hardware and software, such as with application specific integrated circuits. The software can be stored in one or more computers, servers, or other appropriate devices, and can also be kept on a removable storage media, such as a magnetic or optical disks.

### Example 1: CurOnc (fictional)

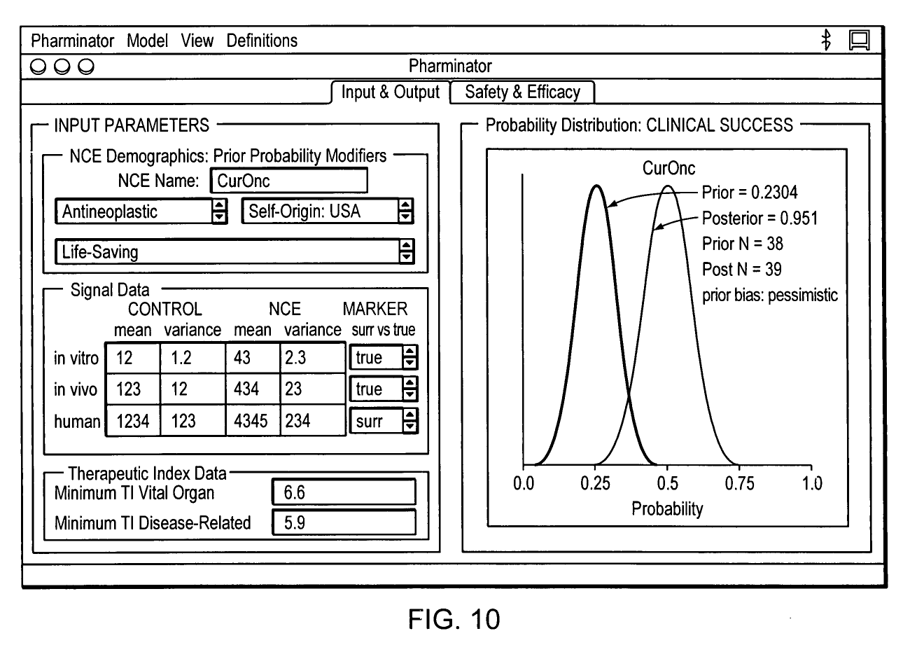

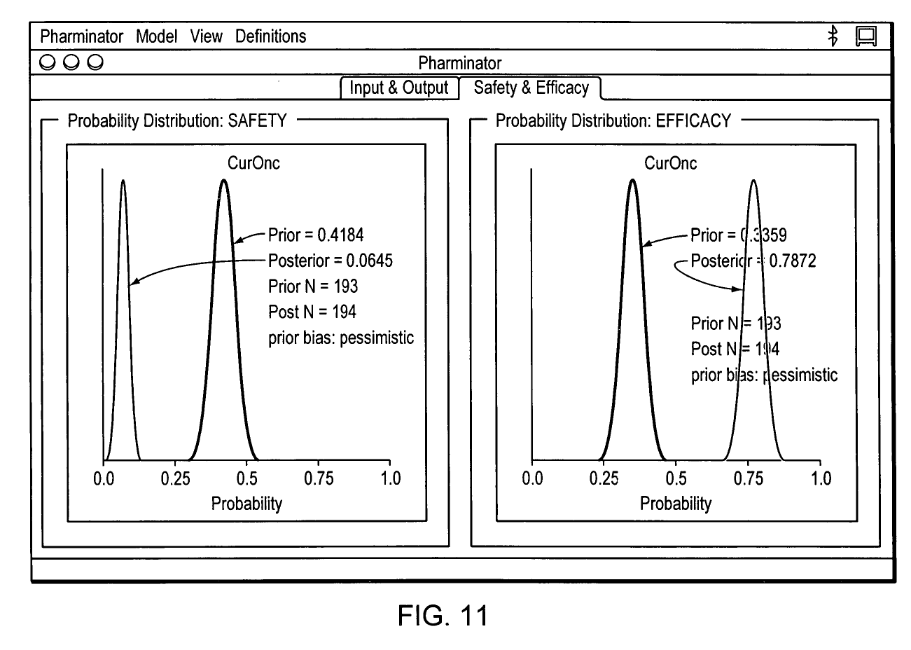

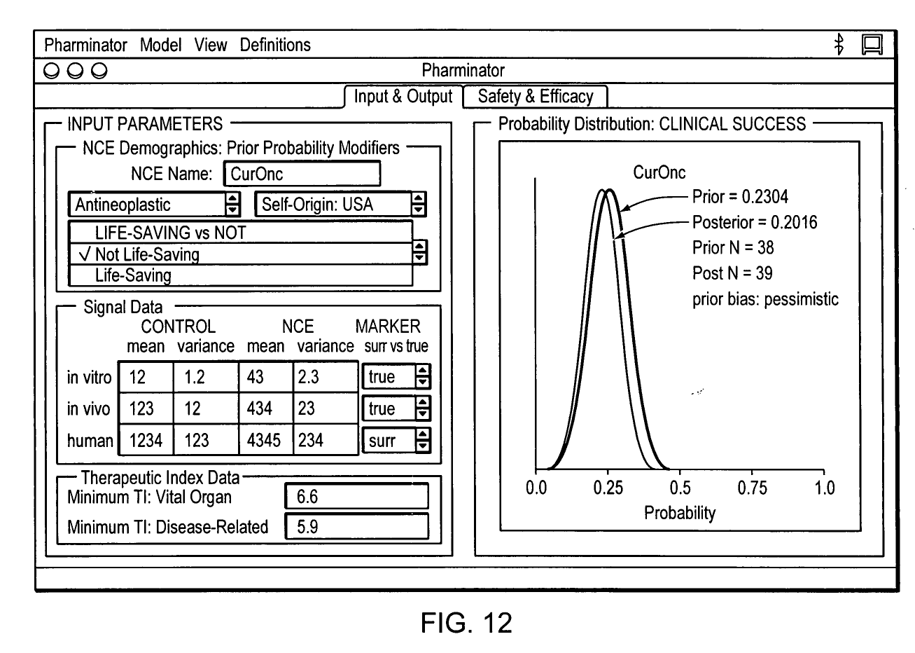

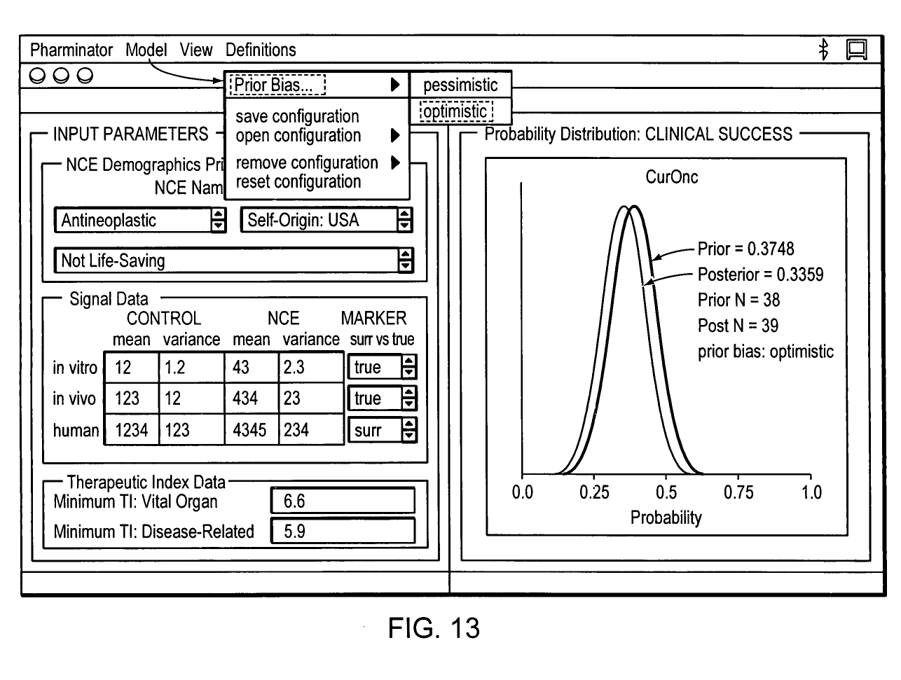

CurOnc is a fictional anti-neoplastic agent devised solely for the purpose of illustrating some key features of Pharminator. CurOnc is self-originated in the USA and meets the definition of “life-saving”. The signal inputs, TI inputs, and Clinical Success probability distribution plots are shown in FIG. 10. The Safety and Efficacy probability distribution plots are shown in FIG. 11. The effect of changing the life-saving option to “Not Life-Saving” is shown in FIG. 12. The effect of changing the prior bias to optimistic is shown in FIG. 13. Overall, the probability distributions generated by Pharminator suggest that CurOnc has a high probability of efficaciousness (0.7872), but is also very likely to have significant toxicity (P(Safety=T)=0.0645). Therefore, if CurOnc is indeed “life-saving”, it has a probability of Clinical Success of 0.4951 with little overlap between the Clinical Success prior (0.2304) and posterior probability distributions. However, if CurOnc is not truly life-saving, its probability of Clinical Success is 0.2016 (less than the prior probability) when prior bias is pessimistic, and at best (prior bias=optimistic), the probability of Clinical Success is 0.3359, which is still less than the prior probability. Given these results, development of CurOnc should be continued only if it is deemed to be truly life-saving.

FIG. 10 is a screen shot of one embodiment according to the invention illustrating the prior and posterior probability distributions for Clinical Success for the fictional antineoplastic agent, CurOnc.

FIG. 11 is a screen shot of one embodiment according to the invention illustrating the prior and posterior probability distributions for Safety and Efficacy for the fictional antineoplactic agent, Cur Onc.

FIG. 12 is a screen shot of one embodiment according to the invention illustrating the effect of selecting the “Not Life Saving” the posterior probability distribution for Clinical Success for the fictional antineoplastic agent, CurOnc.

FIG. 13 is a screen shot of one embodiment according to the invention illustrating the effect of setting the prior bias to “optimistic” on the prior and posterior probability distribution for Clinical Success for the fictional antineoplastic agent, CurOnc.

### Example 2: LY203638 (rhAPC)

Recombinant human activated protein C (rhAPC) is a relatively novel agent that is known for its anti-coagulant, pro-fibrinolytic, and anti-inflammatory properties. Eli Lilly™ Research laboratories has developed LY203638 (rhAPC) as a novel therapy for sepsis (Clinical Investigator's Brochure kindly provided by Dr. Robert Rubin). In general, this example is limited in that several unpublished pre-clinical efficacy studies are listed in the Clinical Investigator's Brochure, but no data are accessible. The most relevant in vitro study was used. This in vitro study was performed prior to the go/no-go decision time point. Bajzar et al reported dose-dependent lysis times, but did not include any measures of variability. Therefore, in vitro variance is set to 0 for both the NCE and control (the in vitro variance entries are actually set to 0.000001 because the program's current implementation will not calculate posterior probabilities if any value is 0. This minor problem will be resolved with future implementations). Published in vivo studies performed prior to the go/no-go decision time point evaluated pre-clinical efficacy in primates, canines, guinea pigs, and rats. The primate data are used for this example because primates are the highest-order species studied. Early phase II study data in humans were provided in the Clinical Investigator's Brochure. This example is an approximation based upon the accessible information only. Note that the true outcome marker is successful treatment of sepsis. The Phase II endpoints reported are therefore all surrogate markers: organ failure-free days, number of transfusion requirements, ICU-, Hospital-, and Ventilator-free days, and 28-day all-cause mortality. For the purpose of this example, organ failure-free days (shock) was chosen as a good sepsis-specific surrogate marker in that multi-organ failure and sepsis-related morbidity are very tightly correlated. No specific data on therapeutic indices could be found either in the Clinical Investigator's Brochure or in the literature from the go/no-go decision time point.

However, the Clinical Investigator's Brochure contains data from Phase I studies at doses ranging from 12-48 μg/kg/hour suggesting that the TI is at least 4 (48/12). Toxicology studies in primates demonstrated that the “no-observed-adverse-effect level” was 2 mg/m²/hour with toxic effects observed at a dose of 8 mg/m²/hour. Taken together, these data suggest that the TI is approximately 4. In the absence of more accurate TI data, this value is used in the example. LY203638 is classified as a cardiovascular agent (since there are no prior data for hematologic agents and it is not antimicrobial). The limitations in acquiring appropriate data for LY203638 underscores the requirement to have unfettered access to the NCE's data in order to optimize Pharminator's predictive accuracy.

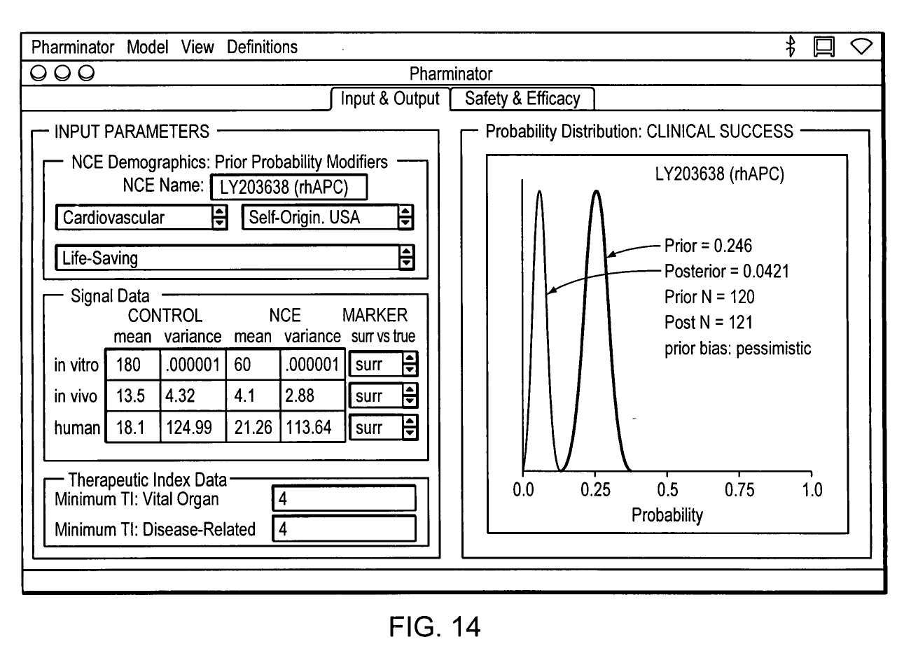

FIG. 14 is a screen shot of one embodiment according to the invention illustrating the prior and posterior probability distributions for Clinical Success for LY203638 demonstrating that it has a very low probability of Clinical Success of 0.0521, much lower than the prior probability of 0.246.

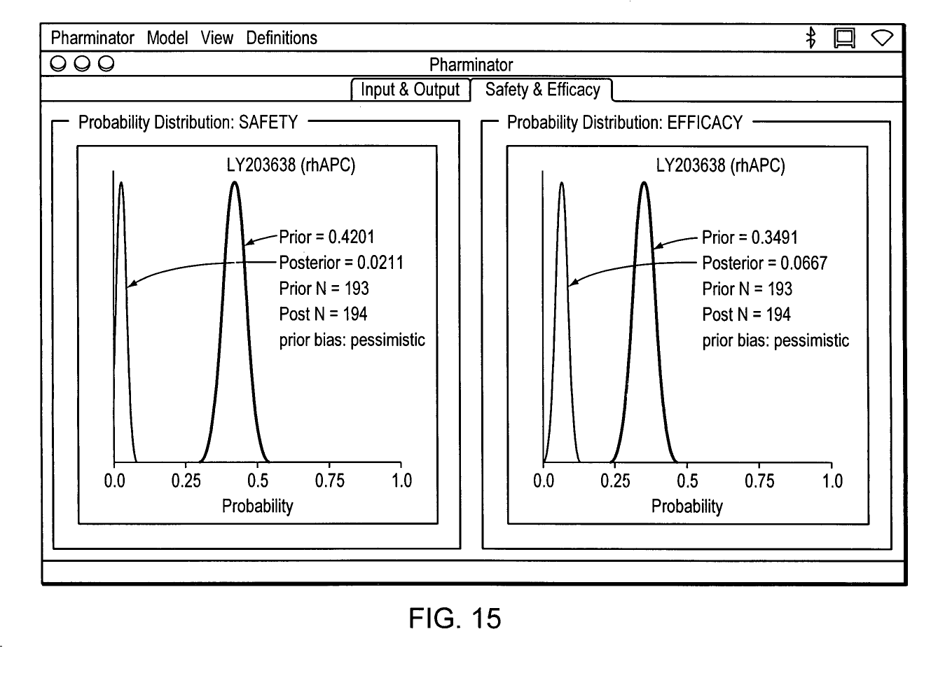

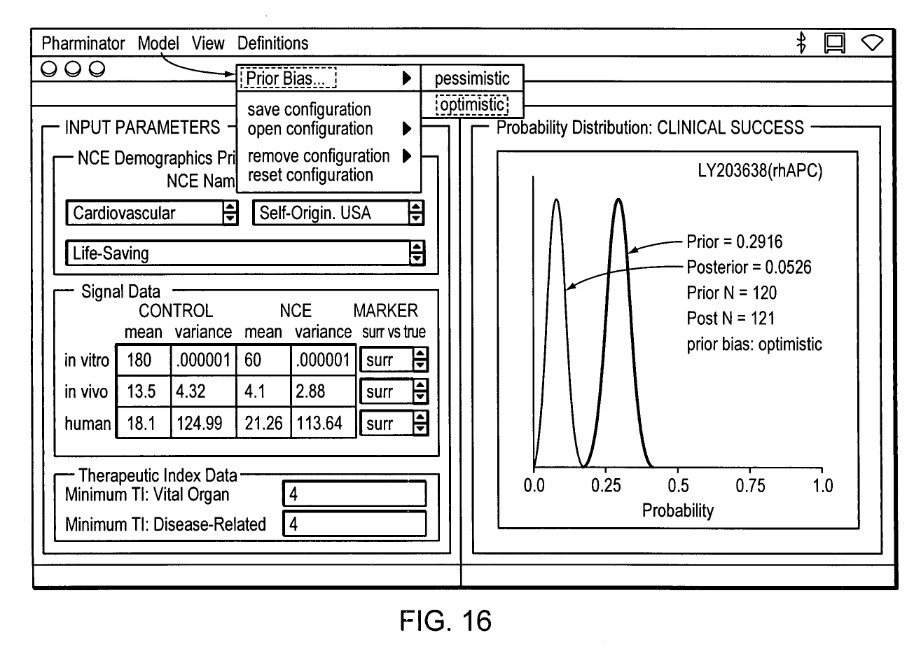

FIG. 15 is a screen shot of one embodiment according to the invention illustrating the prior and posterior probability distributions for Safety and Efficacy for LY203638 (rhAPC) demonstrating that the probabilities of Safety and Efficacy are both very low (0.0211 and 0.0667, respectively). Even when the prior bias is set to optimistic (FIG. 16), the probability of clinical success is only 0.0526, also much lower than the optimistic prior probability of 0.2916.

FIG. 16 is a screen shot of one embodiment according to the invention illustrating the effect of setting the prior bias to “optimistic” on the prior and posterior probability distribution for Clinical Success for LY203638 (rhAPC). Therefore, based only on data available prior to later Phase II studies: even as a life-saving NCE, and when assuming an optimistic prior probability of success, LY203638 has a very low probability of clinical success based only on data available prior to Phase III studies. Of interest, after LY203638 received NDA approval, subsequent post-approval studies raised several concerns about LY203638's safety and efficacy, calling for Phase IV studies to be performed.

It should be appreciated that various aspects of the claimed invention are directed to portions of the systems described, the methods and the processes of the Pharminator embodiments disclosed herein. Further, the terms and expressions employed herein are used as terms of description and not of limitation, and there is no intention, in the use of such terms and expressions, of excluding any equivalents of the features shown and described or portions thereof, but it is recognized that various modifications are possible within the scope of the invention claimed. Accordingly, what is desired to be secured by Letters Patent is the invention as defined and differentiated in the following claims, including all equivalents.

1. A method for predicting the success of a new chemical entity, comprising the steps of:
providing a signal related to a new chemical entity;
providing a therapeutic index for the new chemical entity; and
providing a conditional probability table for the new chemical entity;
providing a prior probability distribution for the new chemical entity;
providing a prior N for the new chemical entity; and
calculating a posterior probability distribution for the new chemical entity.

2. The method of claim 1 wherein the signal is human clinical trial data.

3. The method of claim 1 wherein the signal is in vivo trial data.

4. The method of claim 1 wherein the signal is in vitro trial data.

5. The method of claim 1 wherein the therapeutic index is a vital organ therapeutic index.

6. The method of claim 1 wherein the therapeutic index is a disease therapeutic index.

7. The method of claim 1 wherein the conditional probability table is an efficacy conditional probability table.

8. The method of claim 1 wherein the conditional probability table is a safety conditional probability table.

9. The method of claim 1 wherein the posterior probability distribution is a clinical success posterior probability distribution.

10. The method of claim 1 wherein the posterior probability distribution is an efficacy posterior probability distribution.

11. The method of claim 11 wherein the posterior probability distribution is in response to the pharmacogenomic profile of a patient.

12. The method of claim 1 wherein the posterior probability distribution is a safety posterior probability distribution.

13. An apparatus for predicting the success of a new chemical entity comprising:
an input for a signal related to the new chemical entity;
a therapeutic index for the new chemical entity;
a conditional probability table for the new chemical entity;
a prior probability distribution for the new chemical entity;
a prior N for the new chemical entity; and
a processor calculating a posterior probability distribution for the new chemical entity.

14. The apparatus of claim 14 wherein the signal is human clinical trial data.

15. The apparatus of claim 14 wherein the signal is in vivo trial data.

16. The apparatus of claim 14 wherein the signal is in vitro trial data.

17. The apparatus of claim 14 wherein the therapeutic index is a vital organ therapeutic index.

18. The apparatus of claim 14 wherein the therapeutic index is a disease therapeutic index.

19. The apparatus of claim 14 wherein the conditional probability table is an efficacy conditional probability table.

20. The apparatus of claim 14 wherein the conditional probability table is a safety conditional probability table.

21. The apparatus of claim 14 wherein the posterior probability distribution is a clinical success posterior probability distribution.

22. The apparatus of claim 22 wherein the posterior probability distribution is in response to the pharmacogenomic profile of a patient.

23. The apparatus of claim 14 wherein the posterior probability distribution is an efficacy posterior probability distribution.

24. The apparatus of claim 24 wherein the posterior probability distribution is in response to the pharmacogenomic profile of a patient.
